(19) United States
(12) Patent Application Publication
(10) Pub. No.: US 2021/0236605 A1
(43) Pub. Date: Aug. 5, 2021

(54) COMBINATIONS OF OSTEOPONTIN AND 2'-FUCOSYLLACTOSE FOR USE AS MEDICAMENTS

(71) Applicant: HEALTH AND HAPPINESS (H&H) HONG KONG LIMITED, Hong Kong (CN)

(72) Inventors: Béatrice DUTHEY, Geneva (CH); Jonathan LANE, Cork (IE); Patrice MALARD, Guangzhou (CN); Xiaolei ZE, Guangzhou (CN)

(21) Appl. No.: 17/050,036

(22) PCT Filed: Apr. 24, 2019
(86) PCT No.: PCT/IB2019/000297

§ 371 Date: Oct. 23, 2020

Related U.S. Application Data
(63) Continuation of application No. PCT/IB2018/000464, filed on Apr. 25, 2018.

(51) Int. Cl. A61K 38/39; A61K 31/702; A61P 37/06; A23L 33/17; A23L 33/125; A23L 33/00

(52) U.S. Cl. CPC A61K 38/39; A61K 31/702; A61P 37/06; A23V 2002/00; A23L 33/125; A23L 33/40; A23L 33/17

(57) ABSTRACT

A combination of osteopontin (OPN) and 2′-fucosyllactose (2-FL) is used in the prevention or the treatment of immunological diseases or disorders due to inflammatory factors secretion affecting a human or an animal subject. The said combination is useful, in particular, in the prevention or the treatment of diseases or disorders due cytokine secretion, e.g. INF-γ, IL-2, IL-4, IL-17, IL-6, IL-10, TGF-β, Tbet, GA-TA3 and NFkB and/or immunoglobulin secretion, e.g. IgE and Ig1.

### BACKGROUND OF THE INVENTION

Osteopontin (OPN), which can be highly concentrated in human breast milk, is an extensively phosphorylated acidic glycoprotein that has been associated with the initiation of inflammation, affecting cell adhesion, chemotaxis, immune regulation, and protection against apoptosis, depending on its intracellular or extracellular localization (2-6). Interestingly, OPN has been found to be involved in a number of immune mediated diseases, including multiple sclerosis (7, 8), rheumatoid arthritis (2), systemic lupus erythematosus (3), inflammatory bowel disease (4, 9), asthma (5) and liver disease (10).

Human milk oligosaccharides (HMO) are a family of glycans and the third most abundant fraction in human breast milk (1). The most prevalent HMO, 2′-fucosyllactose (2FL), can account for 30% of all HMOs in the breast milk. This highly bioactive HMO has been associated with immunomodulatory activity, protection against pathogenic bacteria and viruses and improved cognitive function (1).

It has been discovered that these two human breast milk components, that are now commercially available, could positively impact upon immunological disorders which could be due to, but not limited to, modulation of inflammatory cytokine secretion, in particular when used in the form of a combination of OPN and 2-FL wherein the OPN and the 2-FL contents are distinct from the ratio that would be present in any relevant natural source.

Surprisingly, it has been further discovered that OPN and 2-FL, when used in the form of such a combination of OPN and 2-FL could act synergistically to modulate the immune response using a BALB/c murine model of inflammation. It was observed, in particular, that OPN and 2-FL when used in combination could act synergistically to reduce inflammation and regulate immune parameters such as T cell function and cytokine secretion.

These achievements are enabling an unexpected widening of the potential applications of these two components, either as medicaments or in the form of food or nutritional or dietary supplements suited for treating or preventing disorders and diseases initiated by a dysfunction of the immune system of humans or of animals, in particular diseases or disorders due to inflammatory factors secretion.

The invention is defined in the claims as well in the specification appearing below.

### THE INVENTION

A first object of the invention is a combination of osteopontin (OPN) and 2′-fucosyllactose (2-FL) for use in the prevention or the treatment of immunological diseases or disorders due to inflammatory factors secretion affecting a human or an animal subject.

Another object of the invention is a combination of osteopontin OPN and 2′-fucosyllactose (2-FL) for use in the prevention or the treatment of immunological diseases or disorders due to inflammatory cytokine secretion such as IL-17 and IL-4 secretion, or due to immunoglobulin secretion such as IgE secretion affecting a human or an animal subject.

A further object of the invention is a method for preventing or treating immunological diseases or disorders due to inflammatory factors secretion affecting a human or an animal subject, which comprises administering to the subject in need thereof a combination of osteopontin OPN and 2′-fucosyllactose (2-FL), optionally combined with or embedded in a food or nutritional or dietary supplement.

A further object of the invention is a method for preventing or treating immunological disorders due to inflammatory cytokine secretion such as IL-17 and IL-4 secretion, or immunoglobulin secretion such as IgE secretion affecting a human or an animal subject, which comprises administering to the subject in need thereof a combination of osteopontin OPN and 2′-fucosyllactose (2-FL), optionally combined with or embedded in a food or nutritional or dietary supplement.

### Definitions

The term “OPN” defines osteopontin of either human or animal origin as well as any derivative or precursor of same that would exercise the same or equivalent or similar effect when applied within the frame of the invention. This term encompasses an OPN of either human or animal and a recombinant OPN as well.

The term “2-FL” defines 2′-fucosyllactose of either natural, most frequently of mammal origin, of synthetic or of bacterial fermentation origin as well as any derivative or precursor of same that would exercise the same or equivalent or similar effect when applied within the frame of the invention. This term may even encompass, in certain circumstances but still within the frame of the invention, HMOs like 3′-sialyllactose and 6′-sialyllactose.

The term “human subject” is used here to define either pre-terms, newborns, infants, children, teenagers, adults or elderly people, especially infant subjects affected by an immature or dysfunction of their immune function and where the latter needs being restored.

The term “animal subject” defines primarily mammals, like e.g. cattle or pets.

The term “immunological diseases or disorders due to inflammatory factors secretion” encompasses diseases or disorders such as atopic dermatitis, psoriasis, allergy, allergic rhinitis, asthma and chronic obstructive pulmonary diseases (COPD). This enumeration is, however, not limitative.

The term “inflammatory factor” defines cytokines and in particular INF-γ, IL-2, IL-4, IL-17, IL-6, IL-10, TGF-β, Tbet, GATA3 and NFκB, and immunoglobulins, such as IgE and IgG1.

The term “administering” covers either oral or enteral, parenteral or even topical administration.

The terms “combined with” and “embedded in” are common in the art.

### SPECIFIC EMBODIMENTS

### OPN and 2-FL Attenuated the DNCB-Induced AD-Like Symptoms in BALB/c Mice

To investigate the potential therapeutic effect of OPN or 2-FL or an OPN/2FL combination on inflammation and emergence of atopic dermatitis (AD), BALB/c AD model was established by topical application of DNCB on each ear and the dorsal skin. Edema, excoriation, erythema, and scarring were apparent on the skin of DNCB sensitized mice after multiple challenged of DNCB. Strikingly, the severity of DNCB-induced AD-like symptoms in BALB/c mice was ameliorated upon supplementation with OPN (37.5 or 2.7 mg/kg(bw) day) and 2-FL (600 or 75 mg/kg(bw)·day) compared with saline-supplemented mice. Pruritus is an essential feature of AD. The scratching behaviour has already been established as an objective indicator to evaluate pruritus in animal model.

As shown in FIG. 1 oral administration of OPN and 2-FL reduced substantially the AD score. Interestingly, the various combinations of 2FL/OPN, especially those illustrated by trials 7 and 8—demonstrated a significant reduction in AD-like symptoms suggesting a synergistic effect when high doses of OPN are applied.

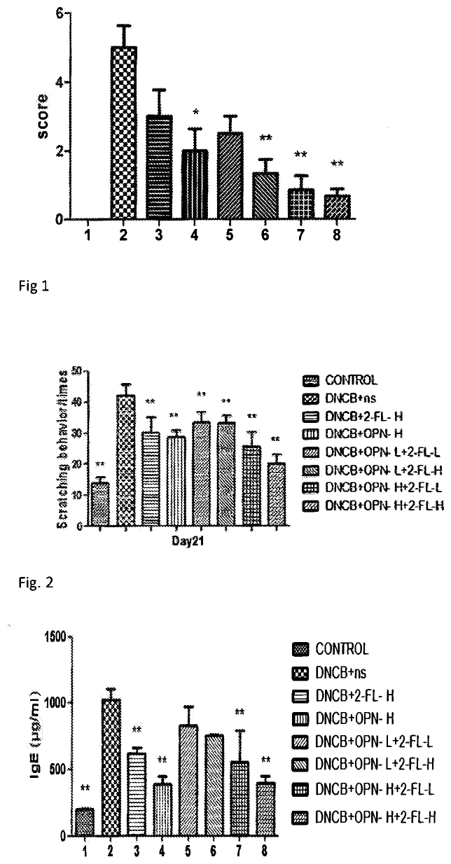

As shown in FIG. 2, oral administration of OPN and 2-FL reduced scratching behaviour. The combination of 2FL/OPN—see in particular trials 7 & 8—demonstrated the most significant reduction in AD-like symptoms suggesting a synergistic effect.

### OPN and 2-FL Reduced Serum IgE Elevation and Alleviated Mast Cells Infiltration in DNCB Sensitized Mice

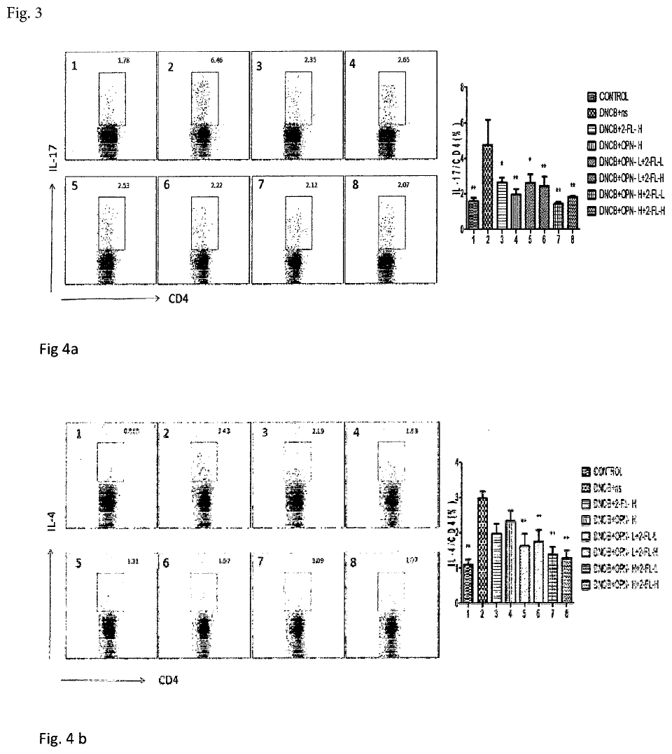

Serum IgE concentrations in mice supplemented with OPN and 2-FL, when compared to the saline control, were determined at day 27. As expected, topical application of DNCB induced a significant expression of serum IgE in BALB/c AD mice (1025.02±82.68 μg/ml), while serum IgE concentrations were decreased in both OPN (388.22±61.28 μg/ml) and 2-FL (621.27±46.79 μg/ml) supplemented groups (FIG. 3).

The high dose combination of OPN/2-FL (396.71±54.71 μg/ml)—see trial 8 in particular—reduced significantly the IgE level, when compared to the other treatments, highlighting a synergistic effect.

Decreased Percentage of Th1, Th2 and Th17 Cells after OPN and 2-FL Treatment in AD-Like Mice

Differentiation of CD4+Th cells in DNCB treated BALB/c mice, with or without 2FL and/or OPN supplementation, were determined. Lymphocytes obtained from DNCB-sensitized mice were tested for the expression of IFN-γ, IL-4 and IL-17 by intercellular staining and subsequently, determined by FACS analysis. The percentage of IFN-γ-producing CD4+Th1 lymphocytes was significantly lower in the OPN and 2-FL supplemented group than that in the saline-treated group. While, the value of IL-4 producing CD4+Th2 lymphocytes and IL-17-producing CD4+Th17 lymphocytes was also significantly decreased in the supplement groups compared with that in the saline-treated group (FIGS. 4(a) and (b)).

These data—see trials 7 & 8 in FIG. 4a as well as trials 6, 7 & 8 in FIG. 4b—suggest that OPN and 2-FL supplementation can decrease substantially AD inflammation by modulating Th1, Th2 and Th17 cell polarization.

OPN and 2-FL Decreased DNCB-Induced mRNA Expression of TSLP and IL-17A in BALB/c Mice

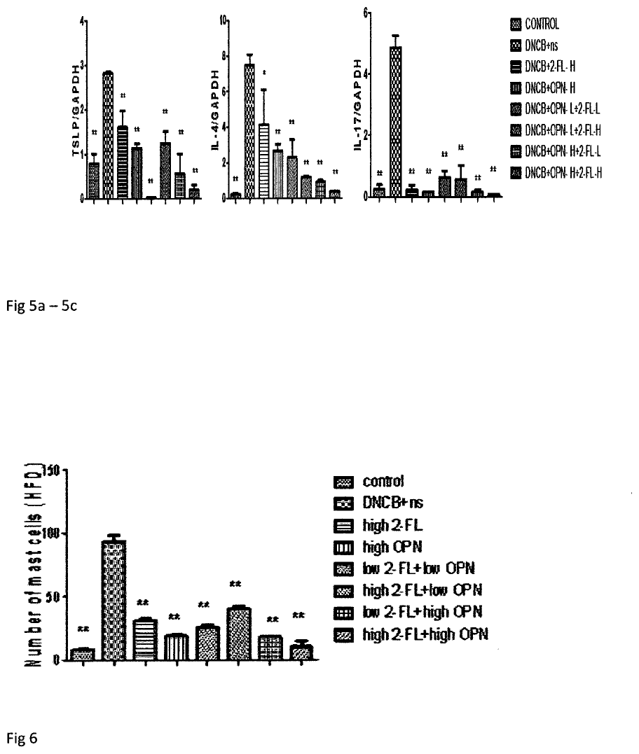

Total RNA was isolated from the dorsal skin of the control and treated BALB/c mice. Subsequently, cytokine mRNA expression in the skin sample was measured by RT-PCR. Expression of Th2-associated cytokines, TSLP and IL-4, were found to be markedly decreased in the OPN and 2-FL-supplemented group compared with the saline-treated group (FIG. 5). Furthermore, the mRNA expression of IL-17 was also significantly lower in skin from OPN or 2-FL supplemented AD mice than that from saline-treated AD mice.

Interestingly, lower expression levels were associated with supplementation of the OPN/2FL combination rather than the individual—ingredients see trials 6 to 8, in particular 7 & 8 in FIG. 5a and FIG. 5b.

### OPN and 2-FL Decrease DNCB-Induced Increases in Th1, Th2 and Th17 Response in Mice

Given that activated CD4⁺ T cells play a crucial role in the pathogenesis of AD, we assayed the subsets of CD4⁺ Th cells in the dLNs of sensitized mice by FACS analysis. The percentage of IFN-γ-producing CD4⁺ Th1 lymphocytes, IL-4-producing CD4⁺ Th2 lymphocytes, IL-17-producing CD4⁺ Th17 lymphocytes was significantly lower in the OPN and 2-FL-treated AD mice than that in the control mice. However, the frequency of Foxp3-positive CD4⁺ Treg lymphocytes were comparable among the groups (FIGS. 8a-8c).

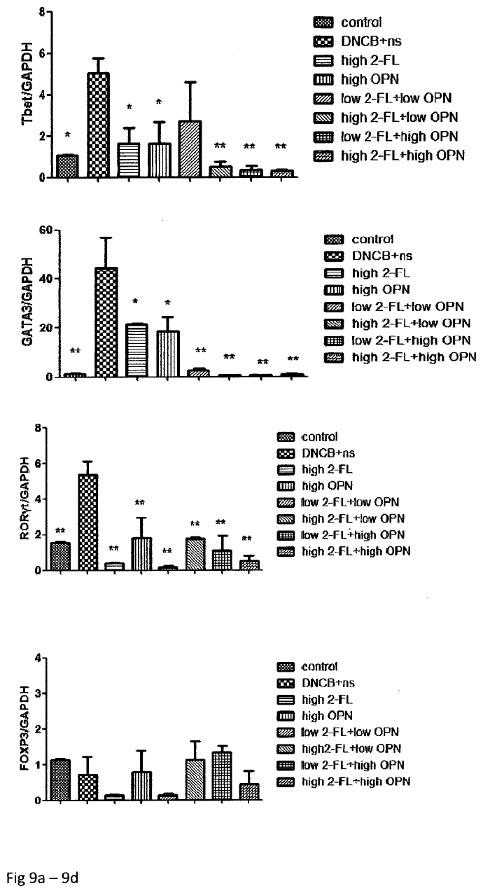

On the other hand, the mRNA level of IFN-γ (Th1 cytokine), IL-4 (Th2 cytokine), and IL-17 (Th17 cytokine) were markedly suppressed in skin lesions from the OPN and 2-FL-treated AD mice compared with those from control AD mice (Figure. 15). Furthermore, we measured the level of the corresponding transcription factors (i.e. T-bet, GATA3, ROR-γt), and the result showed similar changes among the groups (FIG. 9a-9d).

### OPN and 2-FL Inhibit the Infiltration of Mast Cells and Eosinophils to Skin Lesions in DNCB Treated Mice

It is known that mast cells and eosinophils had notoriety based on their detrimental contributions to allergic disorders.

The result showed that the number of mast cells in skin lesions of OPN and 2-FL-treated AD mice was significantly lower than that of WT control mice (FIG. 6).

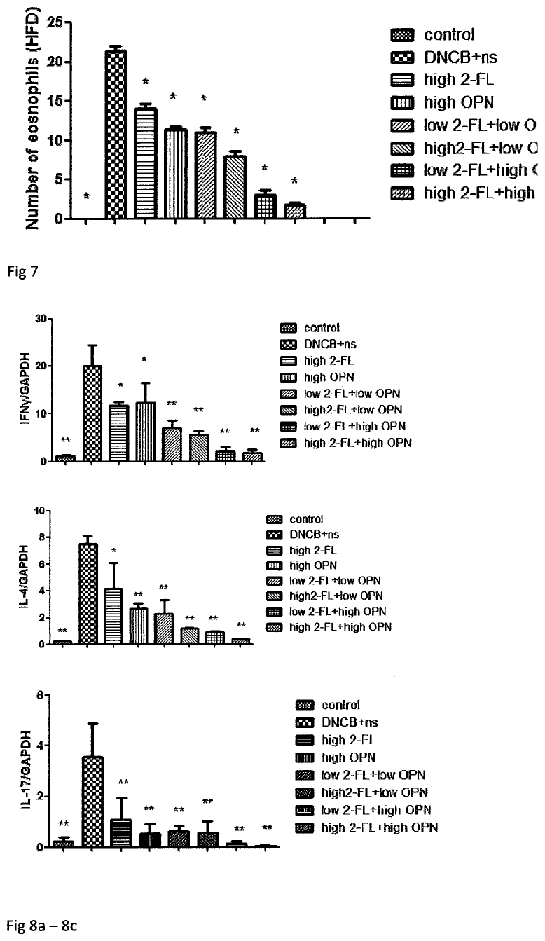

Similarly, the immunohistochemical staining showed that the number of eosinophils infiltrated to the skin lesions of OPN and 2-FL-treated AD mice significantly decreased compared to WT counterparts (FIG. 7).

In general, good results have been achieved by the administration of combinations according to the invention that would provide OPN and 2-FL as well to the subject at a pretty broad dosage of about 2.5 to 45 mg/kg body weight/day and 2-FL at a dosage of about 75 to 750 mg/kg body weight/day.

The best performances, especially those evidencing a synergetic effect (see below in the examples) have been obtained when providing OPN to the subject at high dosages, in particular OPN at a dosage of about 35 to 45 mg/kg body weight/day and 2-FL at a dosage of about 600 mg/kg body weight/day.

As an option and depending on the human subject in need thereof, e.g. an infant, a child or an old person, the medicament comprising the combination of OPN and 2-FL at stake can be administered in combination with or embedded in a food or a nutritional or dietary supplement. This is can apply for oral and enteral administration as well. Suitable food or nutritional or dietary supplements are currently commercially available.

The same would apply to animals like e.g. cattle or pets where applicable.

### 1. Methods

### 1.1 Animal Handling

Balb/c mice were purchased from the Animal Center of Southern Medical University (Guangzhou, China). The animals were maintained under a 12-h light/dark cycle in a specific pathogen-free animal facility at a controlled temperature (20-25° C.) and humidity (50±5%). All mice were fed with regular diet and autoclaved water. All animal experiments in this study were approved by the Welfare and Ethical Committee for Experimental Animal Care of Southern Medical University. To induce AD-like symptom, dinitrochlorobenzene (DNCB) solution (dissolved in a 3:1 mixture of acetone and olive oil) was applied to the dorsal skin and ears of mice (female, 6-8 weeks old). One day after complete dorsal hair removal (approximately 4 cm²), 150 μl of 2% DNCB solution was applied on the dorsal skin, and 10 μl each were applied to the back of both ears at day 1. Four days after sensitization, 0.5% DNCB dissolved in an acetone:olive oil mixture (3:1 vol/vol) is applied to challenge the dorsal skin (150 μl) and the back of both ears (10 μl each) once every two days, (days-27). OPN and 2-FL were dissolved in 0.9% normal saline. Mice treated with DNCB are intragastrically administered with 2-FL, OPN or 2-FL plus OPN from day 0 to day 27. The mice in the vehicle and another DNCB groups are given an equal volume of physiological saline.

### 1.2 Evaluation of AD Score

The severity of dorsal skin lesions were assessed macroscopically according to the following four symptoms: edema, erythema/hemorrhage, excoriation/erosion, and scarring/dryness, and the sum of the individual scores (0, no symptoms; 1, mild; 2, moderate; 3, severe), ranging from 0 to 12, was defined as the final dermatitis scores. These visual assessments were performed every two day and by at least two independent investigators.

### 1.3 Assessment of Scratching Behaviour

Mice were placed into cages for 1 h for habituation. After habituation, the number of scratching episodes for 15 min was counted macroscopically. A series of scratching movements made with the paw was counted as one scratching episode. The total scratching behaviour number was calculated within 15 min. Scratching behaviour was tested at day 7, 14 and day 21 of the experiment.

### 1.4 Determination of Serum IgE Levels

Individual mouse serum was collected on the last day of the experiment. Serum levels of IgE were quantified using the commercial enzyme-linked immunosorbent assay (ELISA) kits (Invitrogen, San Diego, USA) according to the manufacturer's instructions.

### 1.5 Flow Cytometry

Single-cell suspensions from skin draining lymph nodes (dLNs) (axilla and groin) were prepared at the end of the experiment. For Th1, Th2 and Th17 staining, 5×10⁶ lymphocytes were cultured in flat-bottomed 96-well plates in a volume of 500 μl/well with cell stimulation cocktail and protein inhibitor (Invitrogen, San Diego, USA) for 5 h according to the manufacturer's protocol. After surface staining with FITC-labeled rat anti-mouse CD4 (Clone RM4-5, BD Pharmingen, San Jose, Calif., USA), permeabilized cells were stained with PE-Cy7 labeled rat anti-mouse IFN-γ mAb (Clone XMG1.2, BD Pharmingen), APC-labeled rat anti-mouse IL-4 mAb (Clone 11B11, BD Pharmingen). and PE-labeled rat anti-mouse IL-17mAb (Clone eBio17B7, BD Pharmingen).

### 1.6 RNA Isolation and Quantitative Real-Time PCR

Total RNA was isolated from dorsal skin using TRIzol (TransGen Biotech, Beijing, China) according to manufacturer's instruction. 500 ng RNA was quantified for the reverse transcription reaction with TranScript All-in-One First-Strand cDNA Synthesis SuperMix (TransGen Biotech, Beijing, China) in a total volume of 10 μL. Quantitative real-time PCR (RT-PCR) analysis of gene expression was performed using TransStart Green qPCR SuperMix (TransGen Biotech, Beijing, China). The levels of target genes were normalized with respect to GAPDH gene expression.

### 2. Examples(s)

The following examples are a mere illustration of the invention and they are not deemed to represent any restriction of same.

### Example 1

FIG. 1: OPN and 2-FL alleviation of AD-like symptoms induced—as per dermatitis score evaluation—by DNCB in BALB/c mice. (1) control group (2) DNCB group (3) DNCB group+high 2FL (4) DNCB group+high OPN (5) DNCB group+low 2FL+low OPN (6) DNCB group+high 2FL+low OPN (7) DNCB group+low 2FL+high OPN and (8) DNCB group+high 2FL+high OPN. High OPN=37.5 mg/kg(bw)/day, low OPN=2.7 mg/kg(bw)/day, high 2FL=600 mg/kg(bw)/day and low 2FL=75 mg/kg(bw)/day. *p<0.05, **p<0.01.

### Example 2

FIG. 2: The number of scratching episodes for 15 min in BALB/c mice treated with DNCB in the present and absence of OPN and/or 2FL. (1) control group (2) DNCB group (3) DNCB group+high 2FL (4) DNCB group+high OPN (5) DNCB group+low 2FL+low OPN (6) DNCB group+high 2FL+low OPN (7) DNCB group+low 2FL+high OPN and (8) DNCB group+high 2FL+high OPN. High OPN=37.5 mg/kg(bw)/day, low OPN=2.7 mg/kg(bw)/day, high 2FL=600 mg/kg(bw)/day and low 2FL=75 mg/kg(bw)/day

### Example 3

FIG. 3: The level of serum IgE in BALB/c mice treated with DNCB in the presence and absence of OPN and/or 2FL at day 27. **p<0.01. (1) control group (2) DNCB group (3) DNCB group+high 2FL (4) DNCB group+high OPN (5) DNCB group+low 2FL+low OPN (6) DNCB group+high 2FL+low OPN (7) DNCB group+low 2FL+high OPN and (8) DNCB group+high 2FL+high OPN. High OPN=37.5 mg/kg(bw)/day, low OPN=2.7 mg/kg(bw)/day, high 2FL=600 mg/kg(bw)/day and low 2FL=75 mg/kg(bw)/day.

### Example 4

FIGS. 4(a) and (b): Ratio of (a) IL-17-producing CD4+Th17 lymphocytes and (b) IL-4 producing CD4+Th2 lymphocytes in normal saline-treated or OPN and 2-FL-treated AD-like mice (n=6). *p<0.05, **p<0.01. (1) control group (2) DNCB group (3) DNCB group+high 2FL (4) DNCB group+high OPN (5) DNCB group+low 2FL+low OPN (6) DNCB group+high 2FL+low OPN (7) DNCB group+low 2FL+high OPN and (8) DNCB group+high 2FL+high OPN. High OPN=37.5 mg/kg(bw)/day, low OPN=2.7 mg/kg(bw)/day, high 2FL=600 mg/kg(bw)/day and low 2FL=75 mg/kg(bw)/day.

### Example 5

FIGS. 5 (a), (b) and (c): Relative mRNA expression levels of Th2-associated cytokines ((a) TSLP and (b) IL-4) and IL17a, as measured by RT-PCR and expressed as a ratio of GAPDH, extracted from BALB/c mice treated with DNCB in the presence and absence of OPN and/or 2FL. *p<0.05, **p<0.01. (1) control group (2) DNCB group (3) DNCB group+high 2FL (4) DNCB group+high OPN (5) DNCB group+low 2FL+low OPN (6) DNCB group+high 2FL+low OPN (7) DNCB group+low 2FL+high OPN and (8) DNCB group+high 2FL+high OPN. High OPN=37.5 mg/kg(bw)/day, low OPN=2.7 mg/kg(bw)/day, high 2FL=600 mg/kg(bw)/day and low 2FL=75 mg/kg(bw)/day.

### Example 6

FIG. 6: Toluidine blue (TB) staining of skin from DNCB-treated mice was used to identify mast cells. Infiltrations of mast cells in dorsal skin were quantified as means in randomly selected four fields per section (**p<0.01) compared with DNCB+ns group.

### Example 7

FIG. 7: Immunohistochemical staining against eosinophil peroxidase (EPX) was used to identify eosinophils. Infiltrations of eosnophils in dorsal skin were quantified as means in randomly selected four fields per section (*p<0.05) compared with DNCB+ns group.

### Example 8

FIG. 8a-8c: mRNA levels of IFN-γ, IL-4, IL-17 in skin lesions from AD mice with or without 2-FL and OPN treatment were measured by quantitative RT-PCR analysis and expressed as a ratio to GAPDH (*p<0.05, **p<0.01) compared to DNCB+ns group.

### Example 9

FIG. 9a-9d: mRNA levels of T-bet, GATA3, ROR-γt and Foxp3 in skin lesions from AD mice with or without 2-FL and OPN treatment were measured by quantitative RT-PCR analysis and expressed as a ratio to GAPDH (*p<0.05, **p<0.01) compared to DNCB+ns group (FIG. 9).

### REFERENCES

1. Bode, L. (2012) Human milk oligosaccharides: every baby needs a sugar mama. Glycobiology 22, 1147-1162
2. Gravallese, E. M. (2003) Osteopontin: a bridge between bone and the immune system. The Journal of clinical investigation 112, 147-149
3. Kaleta, B. (2014) Role of osteopontin in systemic lupus erythematosus. Archivum immunologiae et therapiae experimentalis 62, 475-482
4. Kourepini, E., Aggelakopoulou, M., Alissafi, T., Paschalidis, N., Simoes, D. C., and Panoutsakopoulou, V. (2014) Osteopontin expression by CD103-dendritic cells drives intestinal inflammation. Proceedings of the National Academy of Sciences of the United States of America 111, E856-865
5. Konno, S., Kurokawa, M., Uede, T., Nishimura, M., and Huang, S. K. (2011) Role of osteopontin, a multifunctional protein, in allergy and asthma. Clinical and experimental allergy: journal of the British Society for Allergy and Clinical Immunology 41, 1360-1366
6. Uede, T. (2011) Osteopontin, intrinsic tissue regulator of intractable inflammatory diseases. Pathology international 61, 265-280

1. A composition comprising osteopontin (OPN) and 2′-fucosyllactose (2-FL) for use in the prevention or the treatment of immunological diseases or disorders due to inflammatory factors secretion affecting a human or an animal subject.

2. (canceled)

3. (canceled)

4. (canceled)

5. The composition according to claim 1 wherein osteopontin (OPN) is of either human or animal origin or recombinant OPN.

6. The composition according to claim 1 wherein 2′-fucosyllactose (2-FL) is selected from the group consisting natural origin, synthetic origin, and bacterial fermentation origin.

7. (canceled)

8. (canceled)

9. The composition according to claim 1 wherein osteopontin (OPN) and 2′-fucosyllactose (2-FL) are in the form of one or several single unit dosage forms.

10. A food or nutritional or dietary supplement comprising the composition according to claim 1.

11. A method for preventing or treating immunological diseases or disorders due to inflammatory factors secretion affecting a human or an animal subject, which comprises administering to the subject a combination of osteopontin (OPN) and 2′-fucosyllactose (2-FL).

12. The method according to claim 11 wherein the inflammatory factor is selected from the group consisting of cytokines and immunoglobulins.

13. The method according to claim 1 wherein the cytokine is selected from the group consisting of INF-γ, IL-2, IL-4, IL-17, IL-6, IL-10, TGF-8, Tbet, GATA3 and NFκB and the immunoglobulin is selected from the group consisting of IgE and IgG1.

14. The method according to claim 10 wherein the immunological disease or disorder is selected from the group consisting of atopic dermatitis, psoriasis, allergy, allergic rhinitis, asthma and chronic obstructive pulmonary diseases (COPD).

15. The method according to claim 10 wherein osteopontin (OPN) is of either human or animal origin or recombinant OPN.

16. The method according to claim 10 wherein 2′-fucosyllactose (2-FL) is of either natural or synthetic origin or from bacterial fermentation origin.

17. The method according to claim 10 wherein osteopontin (OPN) in the combination is administered to the human or animal subject in need thereof at a dosage of about 2.5 to 45 mg/kg body weight/day.

18. The method according to claim 1 wherein 2′-fucosyllactose (2-FL) in the combination is administered to the human or animal subject in need thereof at a dosage of about 75 to 750 mg/kg body weight/day.

19. The method according to claim 1 wherein 2′-fucosyllactose (2-FL) in the combination is administered to the human or animal subject in need thereof at a dosage of about 600 mg/kg body weight/day.
